(19) United States
(12) Patent Application Publication
(10) Pub. No.: US 2018/0085533 A1
(43) Pub. Date: Mar. 29, 2018

(54) Electroformed Needle Cannula

(71) Applicant: Novo Nordisk A/S, Bagsvaerd (DK)

(72) Inventor: Jacqueline Trosborg, Kongens Lyngby (DK)

(21) Appl. No.: 15/562,432

(22) PCT Filed: Mar. 15, 2016
(86) PCT No.: PCT/EP2016/055516

§ 371 Date: Sep. 28, 2017

(30) Foreign Application Priority Data
Apr. 1, 2015 (EP) 15162171.1

(51) Int. Cl. A61M 5/32; C25D 1/00

(52) U.S. Cl. CPC A61M 5/329; A61M 2207/00; C25D 1/00; A61M 5/3286

(57) ABSTRACT

Disclosed herein is a method of electroforming a needle cannula (100) for an injection device, wherein the electroforming method is performed in an electroforming system (1) comprising a cathode (10), an anode (60) and an electrolyte (50) with dissolved metal ions, wherein the method comprises providing a permanent mandrel (10), wherein the mandrel is configured to constitute the cathode. The mandrel (10) comprises a forming portion (20) having a forming surface (21, 22, 23, 24, 25, 26) adapted to form an inner surface of the needle cannula (100), wherein the forming portion (20) comprises a cylindrical axis (A), a longitudinal extension, a first proximal end (16) and a second distal end (17). The method further comprises electrodepositing a metal or metal alloy on the forming surface (21, 22, 23, 24, 25, 26) of the mandrel, where the electrodeposited metal or metal alloy is corresponding to the metal ions dissolved in the electrolyte (50), and whereby the electrodeposited metal or metal alloy is forming a needle cannula (100) on the mandrel (10), and separating the mandrel (10) from the formed needle cannula (100) by moving the mandrel (10) and the electroformed needle cannula relative to each other. Further disclosed is a method of producing different cannula features as composite structures (301, 302, 303, 304, 305) and interlock structures (105, 106, 107, 152, 153).

### TECHNICAL FIELD

The invention relates to a method of producing a needle cannula by electrodeposition. The invention also relates to a needle, wherein the needle cannula is produced by an electrodeposition process.

### BACKGROUND

Needle assemblies are commonly used to either inject of infuse substances into or extract substances out of human or animal bodies. Such needle assemblies are typically disposable and are discarded after only one use.

Traditionally needle cannulas for injection or infusion are produced by drawing a tube down to a desired diameter. However, several down sides are related to this method whereas some of them may be low yield, large requirements to tolerances for production of tubes with small diameters and strong limitations to geometry.

An alternative method of producing microneedles, which may be arranged in arrays, are produces by means of microfabrication processes, where a micromold is fabricated to form the outer surface of the microneedle, and wherein the needle metal is electrodeposited onto the micromold. Such a method is for example described in US 2006/0086689 A1 and US 2002/0138049 A1. Electrodeposition has also been used to produce microneedles for injection devices, where a non-conducting core has been coated with a conducting metal layer. Hereafter, the needle metal is electrodeposited onto the coated layer, and the core is afterwards dissolved in order to produce the final cannula structure. Such a method is for example described in US 2011/0005669 A1, US 2011/0011827 A1 and US 2010/0114043.

US 2007/0256289 A1 describes a method of electroforming an injection needle, by mounting an injection needle master on a master receiving holder, and adhering an electroforming metal to the injection needle master by an electroforming treatment. Finally, the injection needle master is pulled from an electroforming tank together with the master receiving holder whereupon the electroforming body, which constitutes the injection needle main body, is released from the injection needle master. The manufactured injection needle comprises a tapered portion.

JP 2012 005576 A describes a method of manufacturing an injection needle by electrodepositing nickel. Hereafter the injection needle is covered with a photocatalytic coating.

Having regard to the above, it is an object of the present invention to provide an efficient method for producing needle cannulas in a high volume. It is further an object of the present invention to provide a method for efficiently producing needle cannulas with a small diameter, and with fewer limitations to the geometry of the cannula. It is further an object to provide a needle cannula with a small diameter and good flow properties. It is further an object to provide a method of producing needle cannulas with good mechanical properties and with desirable properties with respect to biocompatibility.

### SUMMARY

In the disclosure of the present invention, embodiments and aspects will be described which will address one or more of the above objects or which will address objects apparent from the below disclosure as well as from the description of exemplary embodiments.

In a first aspect a method is provided for electroforming a needle cannula for an injection device, wherein the electroforming method is performed in an electroforming system comprising a cathode, an anode and an electrolyte with dissolved metal ions, wherein the method comprises:
providing a permanent mandrel, wherein the mandrel is configured to constitute the cathode, wherein the mandrel is having a forming portion with a forming surface adapted to form the inner surface of the needle cannula, wherein the mandrel comprises a cylindrical axis, a longitudinal extension, a first proximal end and a second distal end,
electrodepositing a metal or metal alloy on the forming surface of the mandrel, where the electrodeposited metal or metal alloy is corresponding to the metal ions dissolved in the electrolyte, and whereby the electrodeposited metal or metal alloy is forming a needle cannula on the mandrel,
separating the mandrel from the formed needle cannula by moving the mandrel and the electroformed needle cannula relative to each other,
   wherein the system for electroforming comprises a first and a second electrolyte, wherein the first electrolyte comprises a first solution of metal ions, wherein the second electrolyte comprises a second solution of metal ions, wherein the method further comprises:
electrodepositing a first layer of metal or metal alloy corresponding to the metal ions of the first solution onto the mandrel,
electrodepositing a second layer of metal or metal alloy corresponding to the metal ions of the second solution onto the mandrel and/or the first metal or metal alloy.

This method enables a large scale production of thin needless with desired physical, mechanical and biocompatibility properties, which can be obtained by the combination of different metals or alloys each contributing to the overall appearance and functionality of the cannula.

In a further aspect is provided a method of electroforming a needle cannula, wherein the method further comprises forming a composite structure, by electrodeposition of the second layer onto the first layer. In this way is possible to obtain a needle cannula with increased rigidity, and resistance against bending and breakage. It is also possible to cover a first layer by a second layer. The first layer may have desired mechanical properties, but less desired properties with respect to its effect on biological tissue. The second layer covering the first layer may have less desired mechanical properties, but desired properties when in contact with biological tissue are considered. In this way a composite with desired mechanical and biocompatibility properties is obtained.

In a further aspect is provided a method of electroforming a needle cannula, wherein the method further comprises electrodepositing a third layer of metal or metal alloy onto the second layer of metal or metal alloy. The metal of the third layer corresponds to the metal ions of the first solution or to a third electrolyte comprising a third solution of metal ions. Thereby is formed a composite structure, where the second layer is substantially covered or enveloped by the first and the third layer.

In a further aspect is provided a method of electroforming a needle cannula, wherein the first layer of metal or metal alloy and the second layer of metal or metal alloy is electrodeposited at the needle cannula tip, and thereby reinforcing the formed tip in order to reduce the tendency of hooking.

In a further aspect is provided an electroformed needle cannula for an injection device, obtainable by a method as described above, wherein the cannula comprise a first layer of metal or metal alloy, and a second layer of metal or metal alloy, wherein the second layer is deposited at the distal end of the mandrel in order to form a reinforced cannula tip. A reinforced tip will minimize the tendency of hooking.

In a further aspect is proved an electroformed needle cannula for an injection device, obtainable by a method as described above, wherein the cannula comprises a first layer of metal or metal alloy, and a second layer of metal or metal alloy, wherein the outer surface of the needle cannula is covered by the second layer. In a further aspect is proved an electroformed needle cannula, wherein the second layer is a biocompatible outer layer. The outer layer is adapted to be in contact with skin and other biological tissue.

In a further aspect is provided an electroformed needle cannula for an injection device, wherein the needle cannula is formed by a method as described above, wherein the cannula comprises a first biocompatible layer of metal or metal alloy, a second layer of metal or metal alloy, and a third biocompatible layer of metal or metal alloy. In a further aspect is provided a needle cannula, wherein the second layer is covered or enveloped by the first and the third layer.

In another aspect is provided a method of electroforming a needle cannula for an injection device, wherein the electroforming method is performed in an electroforming system comprising a cathode, an anode and an electrolyte with dissolved metal ions, wherein the method comprises:
providing a permanent mandrel, wherein the mandrel is configured to constitute the cathode, wherein the mandrel is having a forming portion with a forming surface adapted to form the inner surface of the needle cannula, wherein the mandrel comprises a cylindrical axis, a longitudinal extension, a first proximal end and a second distal end,
electrodepositing a metal or metal alloy on the forming surface of the mandrel, where the electrodeposited metal or metal alloy is corresponding to the metal ions dissolved in the electrolyte, and whereby the electrodeposited metal or metal alloy is forming a needle cannula on the mandrel,
separating the mandrel from the formed needle cannula by moving the mandrel and the electroformed needle cannula relative to each other,
forming an interlock structure.

The method enables a large scale production of thin needless with desired mechanical properties, which can be obtained by the provision of an interlock structure on the outer surface of the needle cannula. An interlock structure eases the assembly between the injection needle and the needle hub.

In a further aspect is provided, a method of electroforming a needle cannula, wherein the electroforming system further comprises a holding device comprising a local anode, wherein the local anode is adapted to locally increase the deposition rate, and wherein the method further comprises:
positioning the local anode at a desired position relative to the mandrel,
electroforming a needle cannula, wherein the electrodeposition rate is increased at an area in the proximity of local anode, and thereby forms an interlock structure on the needle cannula.

In this way an interlock structure can be produced without further preparation of the needle cannula.

In a further aspect is provided a method of electroforming a needle further comprising:
depositing a conducting material on the forming surface of the mandrel, and thereby creating an interlock forming structure to form an interlock structure on the needle cannula,
forming a needle cannula with an interlock structure, and
separating the needle cannula and the interlock forming structure from the mandrel. In a further aspect is provided a method further comprising removing the interlock forming structure from the needle cannula.

In this way is possible to produce an interlock feature which highly resembles the shape of the interlock forming structure, and it is possible to produce highly detailed interlock structures.

In a further aspect is provided a method of electroforming a needle cannula further comprising:
depositing a polymer on the forming surface of the mandrel, and thereby creating an interlock forming structure to form an interlock structure on the needle cannula,
coating the deposited polymer with a conducting film,
forming a needle cannula with an interlock structure,
separating the needle cannula and the interlock forming structure from the mandrel, and
removing the interlock forming structure from the needle cannula.

The method provides an alternative way of producing cannulas having a highly detailed interlock structure resembling the shape of the interlock forming structure. The interlock forming structure may be easier to remove from the mandrel due to the metal-polymer interface.

In a further aspect is provided a method of electroforming a needle cannula, wherein the electroforming system further comprises a local form giving structure having a forming surface for forming a structure on the needle cannula, and whereby the method further comprises:
positioning the local form giving structure at a desired position relative to the mandrel,
electroforming a needle cannula with a structure corresponding to the forming surface of the local form giving structure, wherein the forming surface of the local form giving structure is adapted to form an interlock structure or a thread on an outer surface of the electroformed cannula.

In a further aspect is provided an electroformed needle cannula for an injection device obtainable by the above described method, wherein the cannula comprises:
an interlock structure adapted to mate with a corresponding structure of a needle hub, and thereby allowing a cannula to snap fit, when inserted into a needle hub.

### BRIEF DESCRIPTION OF DRAWINGS

In the following the invention will be further described with reference to the drawings, wherein

FIG. 1 shows an electroforming system for electroforming a needle cannula

FIG. 2 shows an alternative electroforming system comprising a local form giving structure for electroforming a needle cannula with a sharp tip

FIG. 3 shows an alternative electroforming system comprising a holding device and a local anode for electroforming a needle cannula with an interlock structure

FIG. 4 shows alternative embodiments of a mandrel used in a method for electroforming a needle cannula

FIG. 5 shows alternative mandrels for forming interlock structures.

FIG. 6 shows alternative form releasing structures and local form giving structures used in a method for electroforming a needle cannula

FIG. 7 shows alternative local form giving structures

FIG. 8 shows alternative composite structures produced in a method for electroforming a needle cannula.

### DESCRIPTION

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only.

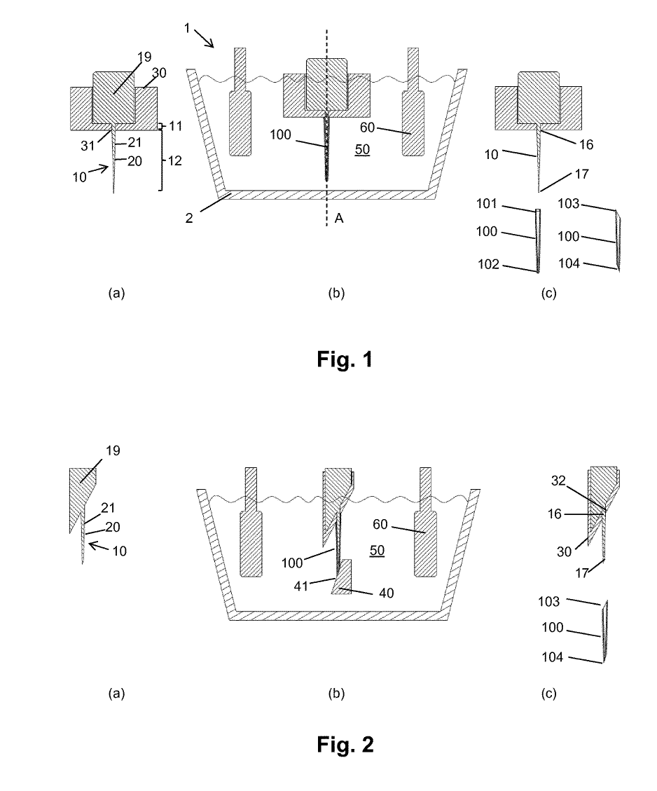

FIGS. 1(a) to 1(c) shows an electroforming system 1, where a needle cannula 100 can be electroformed on a mandrel 10. The electroforming system 1, shown in FIG. 1(b) comprises a mandrel, which can function as a cathode, an anode 60, which may be arranged with rotational symmetry with respect to an axis A, where axis A is a central cylindrical axis of the mandrel. The mandrel comprises a forming portion 20 with a forming surface 21, adapted to form the inner surface of the electrodeposited needle cannula 100. The system further comprises a container 2 containing an electrolyte 50. The electrolyte 50 comprises dissolved metal ions, which may be added by dissolving metal salts or by electrochemically dissolving metal ions from the anode. A current may be driven through the electrolyte, whereby metal ions are taken out of the solution and electrodeposited on the cathode. FIG. 1(a) shows the mandrel 10 and a form releasing structure 30 with a through hole, which may be adapted to snugly fit around the circumference of the mandrel. The form releasing structure 30 can comprise a non-conducting hard polymer. The form releasing structure 30 can be fitted onto the mandrel, whereby the form releasing structure divides the mandrel into a proximal portion 11, which can be covered by the form releasing structure 30, and a distal portion 12, which can be exposed to the electrolyte. During electrodeposition, the mandrel can be immersed into the electrolyte, with the proximal portion shielded from the electrolyte by the form releasing structure 30, and the needle cannula 100 can be formed on the distal portion, which can be exposed to the electrolyte. Therefore, the needle cannula 100 formed on the distal portion 12, can have a larger diameter than the through hole of the form releasing structure 30, and the needle cannula 100, can be released from the mandrel 10, by moving the mandrel 10 and the form releasing structure 30 in opposite longitudinal directions. A form releasing structure 30 can be attached to the mandrel 10, and in this case the forming portion 20 corresponds to the distal portion 12 that can be exposed to the electrolyte 50. The form releasing structure further comprises a proximal form giving structure with a forming surface 31. The naming of the structure is conditioned by the function that it is a structure for giving a form, for example to the proximal end 103 of the needle cannula. As shown in the illustrated case, the forming surface 31 shown in FIG. 1 can be contained in or be approximated with a plane surface having a normal vector, where the vector is parallel to the cylindrical axis A. The forming surface 31 will therefore result in a needle cannula with a surface at the proximal end 101, where a plane containing the form giving surface, or being approximated with the forming surface 31, can have a normal vector essentially parallel to the central axis A. The mandrel 10 further comprises a support structure 19, for manually handling or gripping the mandrel and for making an interface which allows the form releasing structure to hold and support the mandrel 10. If the form releasing structure 30 is not attached to the mandrel 10, the forming surface 21, will correspond to the portion of the mandrel which can be immersed into the electrolyte, and the needle cannula 100 may be separated from the mandrel, by clamping the formed needle cannula 100 with a clamping tool (not shown on figure), and pull it off the mandrel 10. FIG. 1(c) shows that the needle cannula 100 having a proximal end 101 and a distal end 101, can be grinded at both ends in order to produce a sharp proximal tip 103, and a sharp distal tip 104, as in conventional production of needle cannulas with sharp tips.

The above described system 1 enables a method, wherein the electroforming of the needle cannula 100 can be performed in an electroforming system comprising a cathode 10, an anode 60 and an electrolyte 50 with dissolved metal ions. The method comprises, providing a permanent mandrel 10, wherein the mandrel 10 is configured to constitute the cathode 10, wherein the mandrel 10 is having a forming portion 20 with a forming surface 21 adapted to form the inner surface of the needle cannula, wherein the mandrel comprises a cylindrical axis A, a longitudinal extension, a first proximal end 16 and a second distal end 17. Hereafter a metal or metal alloy can be electrodeposited on the forming surface 21 of the mandrel 10, where the electrodeposited metal or metal alloy corresponds to the metal ions dissolved in the electrolyte, and whereby the electrodeposited metal or metal alloy can form a needle cannula 100 on the mandrel 10. The formed needle cannula can be separated from the mandrel, by moving the mandrel and the electroformed needle cannula relative to each other in opposite longitudinal directions. As the mandrel can be a permanent mandrel, and the cannula and the mandrel can be separated without destruction of the mandrel, the method allows an efficient production of electroformed needle cannulas.

FIGS. 2(a) to 2(c) shows an alternative of the system shown in FIG. 1 for describing another aspect of the electroforming system. FIG. 2(a) shows a mandrel 10, where the supporting structure 19 can be adapted to be supported by the form releasing structure 30 shown in FIG. 2(c), where the forming surface 32 of the proximal form giving structure in FIG. 2 can be contained in or be approximated by a plane surface with a normal vector having an angle to the cylindrical axis A, wherein the angle may range from [0-90] degrees, and more preferably from [20-70] deg. The forming surface 32 can therefore form a needle cannula with a surface at the proximal end 101, where a plane containing or being approximated with said surface of the cannula, can have a normal vector having the same angle to the central axis A, as the normal vector of the plane being approximated with or containing the forming surface 32. A method using the system shown in FIG. 2 can therefore produce a needle cannula 100 having a sharp proximal tip 103. Similarly a local form giving structure 40, can be positioned at the distal end of the mandrel. The local form giving structure 40 comprises a form giving surface 41, which can shield a surface at the distal end of the mandrel 10, and thereby create a needle cannula, where the distal tip 104 can be formed by the form giving surface 41. The form giving surface 41 can be approximated with or may be contained in a plane surface with a normal vector having an angle to the central axis A, where the angle may range from [0-90] degrees. If it is desired to produce a flat tip end, the angle should be 0 degrees, and if is desired to produce a sharp tip end 103 the angle may preferably range from [20-70] degrees.

Alternatively or additionally the above described system enables a method, according to another aspect of electroforming a needle cannula for an injection device, wherein the form releasing structure 30 comprises a proximal form giving structure having a forming surface 31, 32 for forming a structure at the proximal end of the needle cannula 100, and whereby the method further comprises electroforming a needle cannula with a proximal tip end 103 corresponding to the forming surface 31, 32 of the form giving structure.

Alternatively or additionally the above described system enables a method, according to another aspect of electroforming a needle cannula for an injection device, wherein the electroforming system further comprises a local form giving structure 40 having a forming surface 41 for forming a corresponding structure on the needle cannula. The local form giving structure 40 may comprise a non-conducting hard polymer, and the method further comprises positioning the local form giving structure 41 at the distal end of the mandrel 10, and thereby electroforming a needle cannula with a structure corresponding to the forming surface of the local form giving structure. In the illustrated case the forming surface is adapted to form a distal tip structure 104 of the electroformed needle cannula 100.

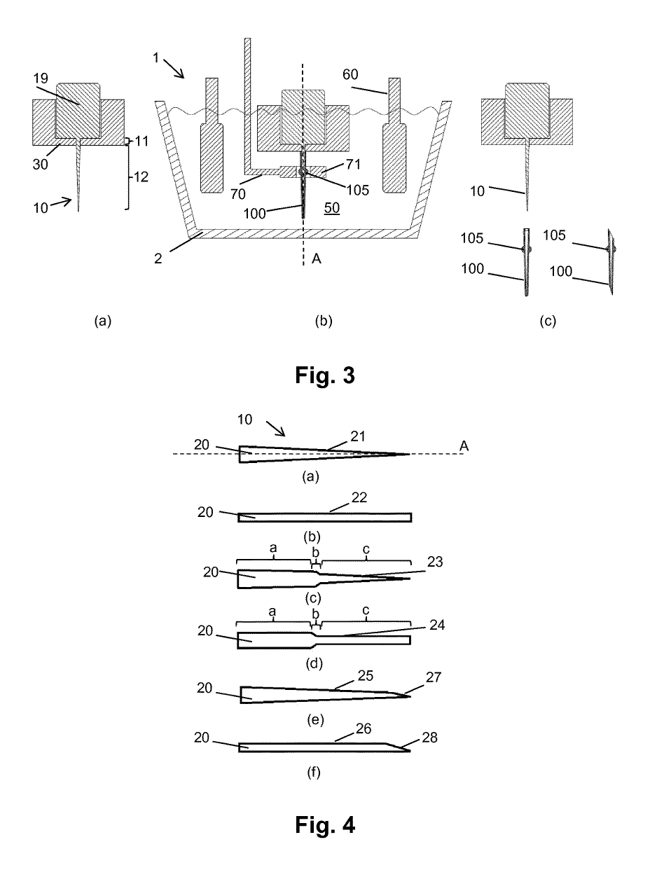

FIGS. 3(a) to 3(c) shows an alternative of the system shown in FIG. 1 for describing another aspect of the electroforming system. FIG. 3(b) shows a system, where a holding device 70 comprises a local anode 71. The local anode can be arranged with rotational symmetry with respect to axis A, and can be positioned at a longitudinal position, where it is desirable to form an interlocking structure.

Alternatively or additionally the above described system enables a method, according to another aspect of the electroforming system. The described system comprises a holding device 70 comprising a local anode 71, wherein the local anode 71 is adapted to locally increase the deposition rate, and wherein the method of electroforming a needle cannula 100 further comprises positioning the local anode at a desired position relative to the mandrel, and electroforming a needle cannula, wherein the electrodeposition rate is increased at an area in the proximity of local anode, and thereby forms an interlock structure on the needle cannula.

### The Mandrel

In one aspect of the electroforming system, it is of importance to produce needle cannulas with high aspect ratios and small diameters, and the internal dimensions of the mandrel can be 0.133 mm, 0.114 mm or 0.089 mm which would correspond to the minimum inner diameters of needle cannulas with normal wall thickness and a gauge size G30, G31, G32 and G33. Needles cannulas with a normal wall thickness and gauge size G32 and G33 both have a minimum internal diameter of 0.089 mm, but it will also be possible to produce cannulas with a smaller gauge size, by using a mandrel with a smaller diameter. In production of mechanical structures with small dimensions, implications due to high aspect ratios (length/width) can be more significant. Similarly are the implications due to the increasing ratios between surface and volume forces more pronounced, i.e., the surface forces will dominate over the volume forces for structures in the micrometer range. The mandrel can have the dimensions, aspect ratios, and surface to volume ratios as shown in the examples in the below table.

| | Corresponding gauge size of needle | | | |
|---|---|---|---|---|
| | with internal diameter corresponding to | | | |
| | the proximal outer diameter of mandrel | | | |
| | G30 | G31 | G32 | G33 |
| Length of mandrel [mm] | 10 | 10 | 10 | 10 |
| Outer diameter at proximal end | 0.133 | 0.114 | 0.089 | 0.089 |
| of mandrel [mm] | | | | |
| Conical surface of mandrel 10⁻⁶ | 2.09 | 1.79 | 1.4 | 1.4 |
| [m²] | | | | |
| Conical volume of mandrel 10⁻¹¹ | 4.63 | 3.4 | 2.07 | 2.07 |
| [m³] | | | | |
| Cylindrical surface of mandrel | 4.18 | 3.58 | 2.8 | 2.8 |
| [m²] | | | | |
| Cylindrical volume of mandrel | 4.63 | 3.4 | 2.07 | 2.07 |
| [m³] | | | | |
| Aspect ratio (Length/Proximal | 75 | 88 | 112 | 112 |
| outer diameter) | | | | |
| Surface to volume ratio (conical) | 45.1 | 52.4 | 67.4 | 67.4 |
| 10³ [m⁻¹] | | | | |
| Surface to volume ratio | 90.2 | 105 | 135 | 135 |
| (cylindrical) 10³ [m⁻¹] | | | | |

Despite the high aspect ratios of the mandrel we have been able to produce needle cannulas with dimensions corresponding to the examples in the above table, and we can therefore produce needle cannulas with internal diameters ranging from 0.070 mm to 0,133 mm, and a wall thickness ranging from 20-150 micrometer and preferably between 30-70 micrometer. In one aspect of the electroforming system, we have been able to use a permanent mandrel, which can be used for the production of several electroformed needle cannulas, and to produce needle cannulas comprising an aspect ratio above 50, and preferably with an aspect above 75. Alternatively, we have been able to use a permanent mandrel, which can be used for the production of several electroformed needle cannulas, to produce needle cannulas comprising a surface to volume ratio above 45×10³ m⁻¹. A preferred result of the method is to produce needle cannulas with a surface to volume ratio above 50×10³ m⁻¹.

In order to improve the separation of the needle cannula from the mandrel, it may be advantageous to provide the mandrel with a thin parting film.

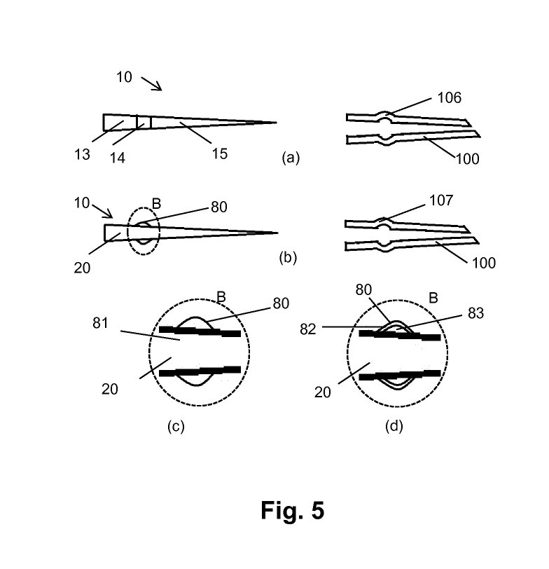

FIG. 4(a) to (f) shows different alternatives of the mandrel 10, which can be made of or may comprise stainless steel, and such mandrels can be produced and formed with high precision and in very small dimensions. The mandrel 10 comprises the forming portion 20 having different forming surfaces 21, 22, 23, 24, 25 and 26, which forms the inner surface of the needle cannula and therefore defines the inner diameter along the needle cannula 100. FIG. 4(a) shows the mandrel corresponding to the mandrel shown in FIGS. 1 to 3.

The alternative embodiments of the mandrel 10 as described above and shown in FIG. 4., are examples of mandrels, where the diameter does not decrease along a longitudinal coordinate running from the left to the right, and where the diameter is defined as a diameter for each cross section of the mandrel. Therefore the mandrels in combination with the systems shown in FIGS. 1 to 3, enables a method according to an aspect of the electroforming method, wherein the diameter is a constant function or an increasing function of a coordinate defined by the cylindrical axis A, and running from the distal end of the mandrel and in the proximal direction. In this way, the diameter function, i.e. a diameter defined as a function of the longitudinal coordinate, enables the separation of the mandrel and the electroformed needle cannula, as no volume of the mandrel is entrapped by the needle cannula. In other words there are no constricting surfaces of a formed needle cannula, that would prevent the separation. The method comprises forming a needle cannula 100 on the mandrel 10, wherein both an outer diameter of the mandrel and an inner diameter of the needle cannula is a constant function or an increasing function of the coordinate defined by the cylindrical axis, and going from the distal to the proximal end.

In one aspect of the electroforming system, the mandrel 10 is a tapered cylindrical beam, as shown in FIG. 4(a), and the forming surface 21 is conical or frustoconical, whereby a formed needle cannula 100, can have an inner surface which is conical or frustoconical surface.

In another aspect of the electroforming system, the mandrel comprises a first section 20a, a second section 20c, and a transition section 20b connecting the first and the second section. Similarly, the forming surface 23, 24 comprises a first forming surface 23a, 24a, corresponding to the first section, a second forming surface 23c, 24c corresponding to the second section, and a transition forming surface 23b, 24b corresponding to the transition section. The transition forming surface 23b, 24b provides a transition surface ensuring a continuous forming surface, when connecting sections with different diameters. The transition section 23b, 24b can connect sections with sloping surfaces or straight surfaces as shown in FIGS. 4(c) and 4(d), but it could also be a combination of sloping and straight surfaces as long as no volume of the mandrel gets in entrapped. Therefore, the diameter of the mandrel 10 is a constant function or an increasing function of a coordinate defined by the cylindrical axis, and running from the distal end of the mandrel and in the proximal direction. The forming surface has a constant slope within each section, but the slope between the sections can be different, and the slope is the spatial derivative of the diameter function. Therefore, the first spatial derivative of the diameter within the first section is a constant, the first spatial derivate of the diameter within the transition section is a constant, and the first spatial derivative of the transition section is larger than the first spatial derivative within the first section. Therefore in an aspect the method further comprises forming a needle cannula comprising a first section, a second section and a transition section corresponding to the forming surface of the mandrel, where the slope of the transition section is larger than the slope of the first section. Alternatively, the transition forming surface comprises a normal vector, and an angle between the cylindrical axis A and the normal vector, wherein the angle is ranging from [0-90] deg.

In one aspect the method further comprises electrodepositing a constant layer on the mandrel, and thereby forming a needle cannula comprising an outer diameter, wherein the outer diameter corresponds to the inner diameter and a constant, and wherein the cannula comprises sections corresponding to the sections of the mandrel. Alternatively, a largest diameter of the second section 23c, 24c is smaller than a smallest diameter of the second section, wherein the method comprises electrodepositing a layer of metal or metal alloy on the mandrel, wherein the cannula comprises sections corresponding to the sections of the mandrel, wherein a largest diameter of the second section is smaller than or equal to a smallest diameter of the first section.

In one aspect of the electroforming system the permanent mandrel comprises an electrical non-conducting polymer coated with a thin electrical conducting film.

In one aspect of the electroforming system, the mandrel comprises a longitudinal variation of the electrical conductivity. The deposition rate is dependent on the electrical conductivity, and increases with increased conductivity of the mandrel, and an alternative of the method is therefore electroforming a needle cannula with a longitudinal variation in the thickness of an electrodeposited layer, where the thickness of the electrodeposited layer corresponds to the electrical conductivity of the mandrel. In this way it would, as an example, be possible to produce a needle cannula 100, where the wall thickness at the proximal end 16 is relatively thicker than at the distal end 17, which would make the cannula stronger in an area where it is connected to a needle hub, and thin at the distal tip, which can penetrate the skin of a patient. As shown in FIG. 5(a), another example is to increase the electrical conductivity at a portion 14 of the mandrel 10, where a corresponding portion of a formed cannula 100, is to form a contact with an inner surface of a through hole in a needle hub. By having a portion 14 with a relatively high electrical conductivity, and portions 13, 15 with a relatively low electrical conductivity, a needle cannula 100 comprising an interlock structure 106 can be formed, i.e., the interlock structure is formed on the portion 14. The electrical conductivity could also be relatively decreased at the portion 14 in order to form a constricting interlock structure on the cannula. The variation of the electrical conductivity along the longitudinal axis can be gradual or more abrupt. It may also be possible to vary the electrical conductivity in a transverse direction, if it is desired to introduce an asymmetric cannula 100.

FIGS. 5(b) and (c) shows another aspect of the electroforming method, where a method of electroforming a needle cannula further comprises forming, depositing or attaching an interlock forming structure 80 on the forming surface 20. The interlock forming structure can be a conducting material 81 attached to the forming surface 20 of the mandrel 10, and thereby creating an interlock forming structure 80 to form a corresponding interlock structure 107 on the needle cannula 100. The deposited conducting material 81 can be removably deposited, which enables the deposited conducting material to be separated from the mandrel along with a formed needle cannula 100. After the needle cannula 100 and the conducting material 81 has been separated from the mandrel, the interlock forming structure 80 can be removed from the cannula by mechanical means or by chemically dissolving the material. The interlock forming structure can as an example be formed as a continuous protruding ring or distributed domes. In order to ensure a sufficient interlock, the distributed domes can be arranged with rotation symmetry to the axis A.

As shown in FIGS. 5(b) and (d), the interlock forming structure 80 can, alternatively, be created by attaching a polymer 83 on the forming surface 20 of the mandrel 10. Hereafter, the deposited polymer is coated with a conducting film 82, in order to create the interlock forming structure 80, which thereby enables a method to form the interlock structure 107 on the needle cannula 100. Afterwards, the needle cannula 100 and the interlock forming structure 80 are separated from the mandrel 10, by pulling the mandrel and the needle cannula 100 in opposite direction. Additionally, the interlock forming structure 80 can be removed from the needle cannula 100 by mechanical or chemical means.

Alternatively, the mandrel can comprise a flexible but permanent polymer core comprising a thin conducting layer. The interlock forming structure 80, can be strongly bonded to or integrated with the conducting layer, and the interlock forming structure 80 can be adapted to flex towards the cylindrical axis, when a radial force is acting on the interlock forming structure 80. As an example, the interlock forming structure 80 can be formed as distributed domes. Due to the flexibility and compressibility of the core, and the special configuration of the interlock forming structure 80, the permanent mandrel can be separated from a formed cannula, as the separation will flex or push the forming structure 80 in a radial direction towards the axis A.

### Anode

In one aspect of the electroforming system, the anode 60 comprises a metal corresponding to the metal deposited on the mandrel, and thereby the method further comprises continuously replenishing the metal used for deposition, by dissolving metal from the anode.

### Form Releasing Structure

In an aspect of the electroforming system, the system comprises a form releasing structure 30, the form releasing structure 30 comprises at least one through hole adapted to receive the mandrel, and the mandrel is inserted into the through hole. The form releasing structure can divide the mandrel into a distal portion and a proximal portion, and the distal portion can be exposed to the electrolyte. The proximal portion is to be shielded against the electrolyte. The form releasing structure can be used to separate the mandrel 10 from the formed needle cannula 100 by moving the mandrel 10 and the form releasing structure 30 in opposite directions. The form releasing structure can comprise a non-conducting hard polymer.

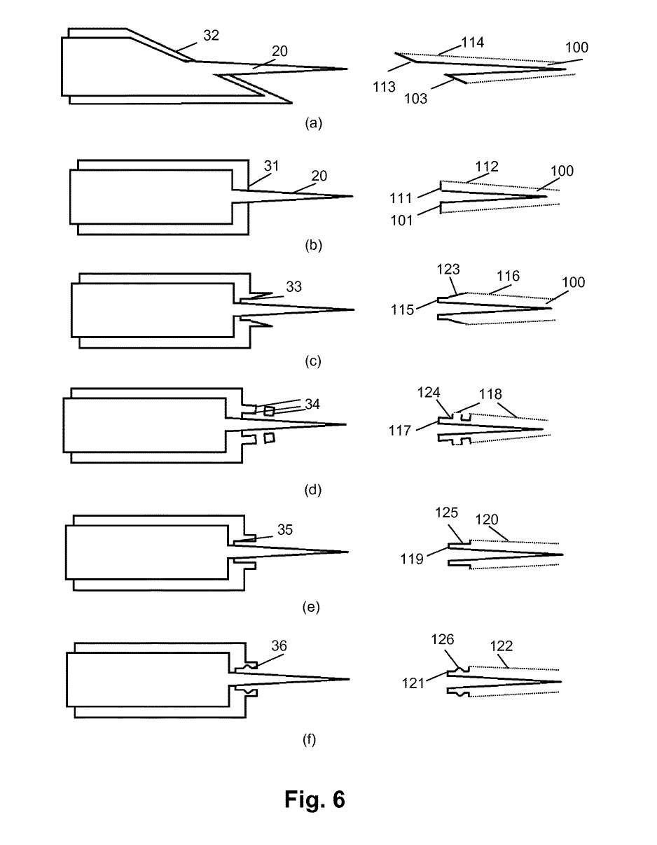

FIG. 6(a) to FIG. 6(f) shows different alternatives of an aspect of the electroforming system, wherein the form releasing structure 30 comprises a proximal form giving structure having a forming surface 31, 32, 33, 34, 35, 36 for forming a structure at the proximal end of the needle cannula 100, and whereby the method further comprises electroforming a needle cannula with a structure corresponding to the forming surface 31, 32, 33, 34, 35, 36 of the form giving structure. FIGS. 6(a) to 6(f) also illustrates the formed surfaces on the needle cannula 100, where the solid lines 111, 113, 115, 117, 119, 121 illustrates the inner surfaces formed by the forming surface 21 of the mandrel 10, and the outer surfaces formed by the forming surface 31, 32, 33, 34, 35, 36 of the form giving structure. The dotted lines 112, 114, 116, 118, 120, 122 illustrates the free surfaces, which can grow freely in the electrolyte solution. FIG. 6 shows by example the use of forming surface 21, but mandrels with other forming surfaces can also be used, e.g., the mandrels with the forming surfaces shown in FIG. 5. FIGS. 6(a) and 6(b) shows the alternative embodiment, which also has been shown in FIGS. 1 to 3, where the forming surfaces 32, 31 can form the proximal tip 103, 101 of the needle cannula 100.

In an aspect of the electroforming system, the forming surface 31, 32, 33, 34, 35, 36 of the proximal form giving structure is adapted to form an outer surface 111, 113, 115, 117, 119, 121 and/or a proximal tip structure 101, 103 of the electroformed cannula. In all the cannulas illustrated in FIG. 6, the formed surfaces comprises a proximal tip structure, and the surface of the proximal tip structure is a part of the outer surface 111, 113, 115, 117, 119, 121, the proximal tip structures are not specifically numbered in FIG. 6(c) to FIG. 6(f). The surface of the proximal tip structure 101, 103 is a portion of the outer surface 111, 113.

In an aspect of the electroforming system, the forming surface 34, 36 of the proximal form giving structure is adapted to form an interlock structure or a thread on an outer surface 117, 119 of the electroformed cannula. FIG. 6(d) shows that an interlock structure 124 can be formed by a proximal form giving structure, where distributed openings or a ring opening allows the electrolyte to enter into the proximal form giving structure. The formed metal can grow into the openings and form an interlock structure 124 on the outer surface 117. The needle cannula and the mandrel are separated by pulling the form releasing structure 30 and the mandrel in opposite directions, and afterward the form releasing structure 30 and the proximal form giving structure can be opened in order to release the formed and separated needle cannula. Similarly, distributed indents or a ring shaped indent in the forming surface 36 shown in FIG. 6(f), can form an interlock structure 126 on the outer surface 119.

In an aspect of the electroforming system, the forming surface 31, 32, 33, 34, 35, 36 of the proximal form giving structure comprises a conducting film coating, and in this way the forming surface 31, 32, 33, 34, 35, 36 of the proximal form giving structure can constitute a cathode of the electroforming system.

In an aspect of the electroforming system, the conducting film coating is adapted to be separated from the forming surface 31, 32, 33, 34, 35, 36 of the proximal form giving structure, and wherein the method further comprises separating the electroformed needle cannula from the forming surface 31, 32, 33, 34, 35, 36 of the proximal form giving structure, by separating the conducting film from the forming surface of the proximal form giving structure.

In an aspect of the electroforming system, the proximal form giving structure can comprise a channel, or a number of small channels to allow a fresh supply of electrolyte and dissolved metal ions to the proximal end of the mandrel and the forming surface 33, 34, 35, 36 of the proximal form giving structure. In this way the channels ensure that the used metal ions are replenished.

### The Local Form Giving Structure

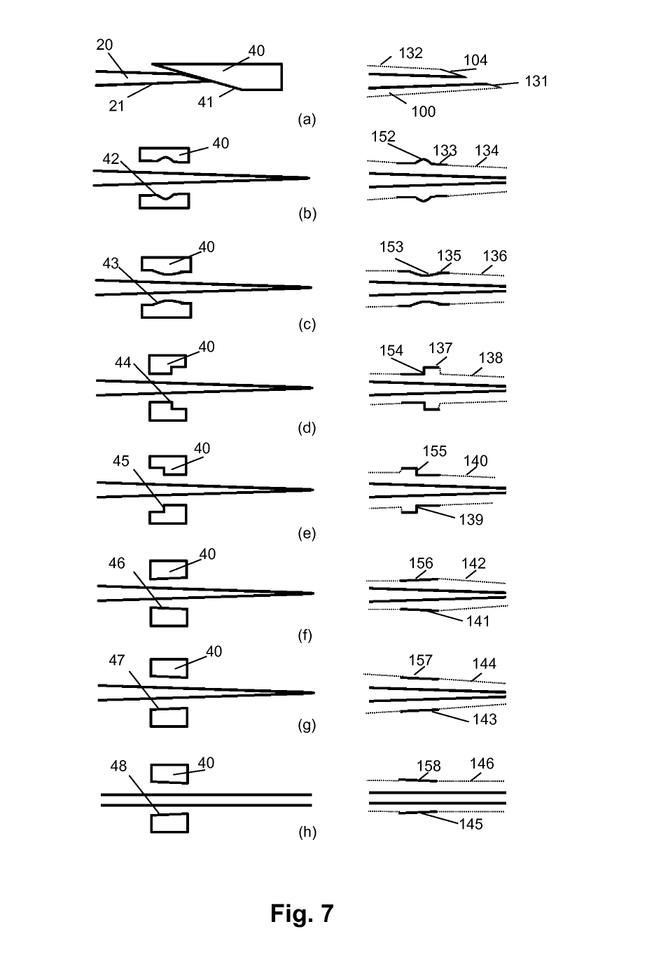

FIG. 7(a) to FIG. 7(h) shows the local form giving structure 40 positioned at different positions relative to the mandrel, and wherein the form giving structure 40 comprises alternative forming surfaces 41, 42, 43, 44, 45, 46, 47, 48 to form an outer surface 131, 133, 135, 137, 139, 141, 143, 145 on the needle cannula 100. FIG. 7 illustrates the cannula resulting from an electroforming process, where the illustrated mandrel and local form giving structure can be used. For the illustrated cannula 100, the solid lines indicates the surface formed by the mandrel, and the surface 131, 133, 135, 137, 139, 141, 143, 145 formed by the forming surface 41, 42, 43, 44, 45, 46, 47, 48 of the local form giving surface. The dotted lines illustrated the free surfaces 132, 134, 136, 138, 140, 142, 144, 146 formed freely in the electrolyte.

FIG. 7(a) illustrates the formation of a distal needle tip 104, and as also illustrated in FIG. 2. Although not illustrated in the figure the local form giving structure can also produce a flat distal tip by providing a forming surface where the normal vector is parallel to the cylindrical axis A. FIG. 7(b) illustrates the formation of a cannula 100 comprising a protruding interlock structure 152 formed on the surface 133, where the interlock for example can be symmetrically distributed domes or a ring shaped protrusion. FIG. 7(c) shows a cannula 100 comprising a constricting interlock structure 153 formed on surface 135. FIG. 7(d) shows an insertion stop 154 formed on the surface 137, where the needle cannula 100 is adapted to be inserted into a through hole of a needle hub until the insertion stop 157 abuts with a corresponding structure of the needle hub. FIG. 7(e) similarly shows an insertion stop 155 adapted to be inserted into a needle hub, however, the cannula illustrated in FIG. 7(e) is to be inserted from the opposite side of the hub, as compared to the cannula illustrated in FIG. 7(d). FIG. 7(f) shows a cannula comprising a pressure fit structure 156 formed on the surface 141, where the cannula is adapted to pressure fit into a needle hub with a corresponding surface. The pressure fit structure 157 shown in FIG. 7(g) is for insertion from the opposite direction compared to the cannula shown in FIG. 7(f). FIG. 7(h) shows a pressure fit on a straight needle cannula, and the figure illustrates the above shown structures also can be formed on straight cannulas. The formation of cannulas with insertion stop structures and pressure fit structures minimizes the requirements to the tolerances of the produces cannulas. Although not illustrated, the forming surface can also comprise a thread, which can be formed onto the outer surface of the needle cannula.

In one aspect of the electroforming system further comprises a local form giving structure 40 having a forming surface 41, 42, 43, 44, 45, 46, 47, 48 for forming a structure on the needle cannula, and whereby the method further comprises positioning the local form giving structure 40 at a desired position relative to the mandrel 10, and electroforming a needle cannula 100 with a structure corresponding to the forming surface 41, 42, 43, 44, 45, 46, 47, 48 of the local form giving structure 40. The local form giving structure 40 can comprise a non-conducting hard polymer.

In an aspect of the electroforming system, the forming surface 41, 42, 43, 44, 45, 46, 47, 48 of the local form giving structure 40 is adapted to form an outer surface 131, 133, 135, 137, 139, 141, 143, 145 and/or a distal tip structure 104 of the electroformed cannula. The surface of the distal tip structure 104 is a portion of the outer surface 131.

In an aspect of the electroforming system, the forming surface 42, 43 of the local form giving structure 40 is adapted to form an interlock structure 152, 153 or a thread on an outer surface of the electroformed cannula 100.

In an aspect of the electroforming system, the forming surface of the local form giving structure comprises a conducting film coating. The forming surface 41, 42, 43, 44, 45, 46, 47, 48 of the local form giving structure constitutes a cathode of the electroforming system.

In an aspect of the electroforming system, the conducting film coating is adapted to be separated from the forming surface 41, 42, 43, 44, 45, 46, 47, 48 of the local form giving structure, and wherein the method further comprises separating the electroformed needle cannula 100 from the forming surface 41, 42, 43, 44, 45, 46, 47, 48 of the local form giving structure, by separating the conducting film from the forming surface 41, 42, 43, 44, 45, 46, 47, 48 of the local form giving structure.

In an aspect of the electroforming system, the local form giving structure 40 comprises one or more channel to allow a fluid flow and to allow the electrolyte and the dissolved metal ions to replenish the metal deposited at the position of the local form giving structure.

In one aspect of the electroforming system, the method of using the system allows for the formation of a needle cannula comprising an interlock structure 152, 153, an insertion stop 154, 155 or a pressure fit structure 156, 157, 158.

### The Holding Device

The system can comprise a holding device 70 as illustrated on FIG. 2(a) comprising a local anode 71. Therefore, in one aspect, the system comprises a holding device 70 comprising a local anode 71, wherein the local anode 71 is adapted to locally increase the deposition rate, and wherein the method further comprises positioning the local anode at a desired position relative to the mandrel. The method also comprises electroforming a needle cannula, wherein the electrodeposition rate is increased at an area in the proximity of the local anode 71, and thereby forms an interlock structure 105 on the needle cannula.

In another aspect of the electroforming system, the holding device comprises one or more channels to allow a fluid flow and to allow the electrolyte and the dissolved metal ions to replenish the metal deposited at the position of the local anode 71.

In another aspect of the electroforming system, the anode comprises a metal corresponding to the metal deposited on the mandrel 10, and thereby the method further comprises continuously replenishing the metal used for deposition, by dissolving metal from the anode 71.

### Metals for Electroforming

In one aspect of the electroforming system, the electrodeposited metal can be Cr, Mn, Tc, Re, Fe, Ru, Os, Co, Rh, Ir, Ni, Pd, Pt, Cu, Ag, Au, Zn, Cd, Hg, In, Tl, Sn, Pb, As, Sb, Bi, Se or Te.

In another aspect of the electroforming system, the electrodeposited metal alloy is cobalt based alloys, nickel based alloys, iron based alloys, gold based alloys, silver based alloys or platinum based alloys.

More specific examples of alloys can be CoNi, CoCrMo, CoSn, NiSn, NiW, NiTi, FeNiCr, FeCr, FeNiCrMo, PtIr

In another aspect of the electroforming system, the electrodeposited metal alloy comprises one of the elements V, Nb, Mo, W, B, Al, Ge or P.

### The Composite Structure

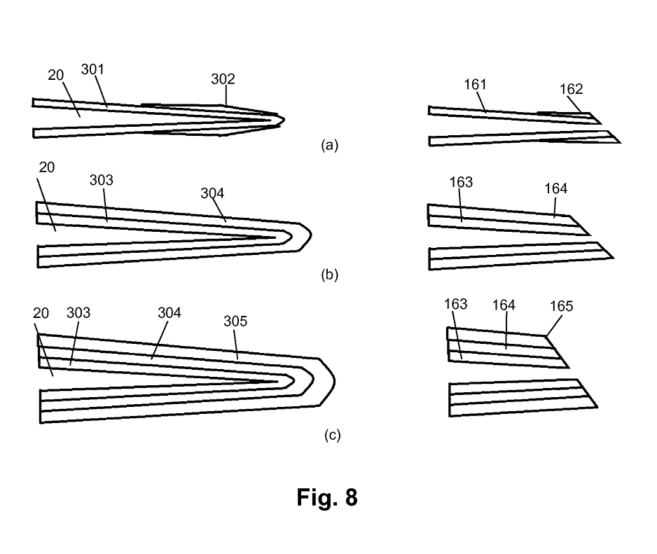

One advantage of electroforming is the ability to form composite structures by forming layered structures comprising different metal or metal alloys. In this way it is possible to combine desired properties of one material with desired properties of another material. FIGS. 8(a) to 8(c) shows a mandrel 10 comprising a forming portion 20, whereupon different layers of metal and metal alloy can be formed. FIG. 8(a) also illustrates the resulting cannulas. FIG. 8(a) illustrates the formation of a first layer 301, on the forming portion 20, and the formation of a second metal 302 on a portion of the first metal. The formed metals does not necessarily extend along the entire length of the forming portion. FIG. 8(a) also illustrates, as an example, a formed cannula comprising a first metal 161, and a second metal 162 increasing the mechanical strength of the tip. FIG. 8(b) shows the formation of a first layer 303 on the forming portion 20, and the formation of a second layer 304 on the first layer 303. FIG. 8(b) also shows, as an example, a needle cannula comprising two layers 163, 164. FIG. 8(c) shows the additional formation of a third layer 305 on top of the second layer 304, and the resulting composite needle cannula comprising three layers 163, 164, 165. The second layer 164 is essentially covered by the first 163 and the third layer 135.

Therefore, in one aspect the system for electroforming comprises a first, and a second electrolyte, wherein the first electrolyte comprises a first solution of metal ions, wherein the second electrolyte comprises a second solution of metal ions, wherein the method further comprising:
electrodepositing a first metal or metal alloy 301 corresponding to the metal ions of the first solution onto the mandrel 10,
electrodepositing a second metal or metal alloy 302 corresponding to the metal ions of the second solution onto the mandrel 10 and/or the first metal or metal alloy 301. By this method it is for example possible to produce a needle cannula with an increased mechanical strength at the distal tip by applying a second metal or metal alloy at the distal end of the mandrel, where the second metal or metal alloy has desired mechanical properties, i.e., sufficient mechanical strength.

In another aspect, the method further comprises electrodepositing a first layer 303 of metal or metal alloy corresponding to the metal ions of the first solution onto the mandrel, electrodepositing a second layer 304 of metal or metal alloy corresponding to the metal ions of the second solution onto the first layer of metal 303 or metal alloy, and thereby forming a needle cannula comprising a composite structure.

In another aspect, the method further comprises electrodepositing a third layer 305 of metal or metal alloy onto the second layer 304 of metal or metal alloy, wherein the metal of the third layer corresponds to the metal ions of the first solution or a third electrolyte comprising a third solution of metal ions, and thereby forming a needle cannula comprising a composite structure, where the second layer 304 is substantially covered by the first and the third layer.

In another aspect, the first metal or metal alloy and 301, and/or the second metal or metal alloy 302 is electrodeposited at the needle cannula tip, and thereby reinforcing the formed tip in order to reduce the tendency of hooking.

### Other Process Steps and Parameters

In one aspect, the method further comprises using additives, electrochemical feedback or pulse power and thereby reducing stress in the electroformed metal or metal alloy.

In one aspect, the method further comprises optimizing an applied voltage in order to optimize quality and production speed.

In one aspect, the method further comprises performing a finishing process on the electroformed cannula, where the finishing process comprises grinding of the needle tip.

In one aspect, the method further comprises rinsing and degreasing the mandrel before electrodeposition. The mandrel can for example be degreased in NaOH.

In one aspect, the method further comprises forming a plurality of needle cannulas by parallel processing of the mandrels.

In one aspect, the form releasing structure comprises an array adapted to arrange an hold the mandrels when they are processed.

### Examples of Formed Products

The above described methods can be used to form various alternative embodiments of needle cannulas, and such cannulas will be exemplified in the following.

In one aspect, the products comprises to an alternative embodiment of an electroformed needle cannula for an injection device, wherein the cannula comprises a wall thickness ranging from [20-150] micrometer or more preferably from [30-70] micrometer.

In another aspect, the products comprises an alternative embodiment of an electroformed needle cannula for an injection device comprising an outer diameter of the distal tip ranging from [120-270] micrometer or more preferably from [170-270] micrometer.

In another aspect, the products comprises an alternative embodiment of an electroformed needle cannula for an injection device comprises, a first section having a first outer diameter, a second section having a second outer diameter and a transition section, wherein the transition section connects the first and the second section, and wherein the first outer diameter is different from the second outer diameter, wherein the transition section is adapted to define an insertion stop, for a cannula inserted into a needle hub.

In another aspect, the products comprises to an alternative embodiment of an electroformed needle cannula for an injection device comprising a first section having a first diameter, a second section having a second diameter and a transition section, wherein the transition section connects the first and the second section, and wherein the first diameter is different from second diameter, wherein the surface of the transition section is tapered and thereby allows the cannula to be pressure fitted, when inserted into a needle hub.

In another aspect, the products comprises an alternative embodiment of an electroformed needle cannula for an injection device comprising an interlock structure 105, 106, 107, 124, 126, 152, 153 adapted to mate with a corresponding structure of a needle hub, and thereby allowing a cannula to snap fit, when inserted into a needle hub, wherein the interlock structure forms a protrusion on the outer surface of the needle cannula, or wherein the interlock structure 153 forms a constriction in the outer surface.

In another aspect, the products comprises an alternative embodiment of an electroformed needle cannula for an injection device, wherein the cannula comprises an interlock structure adapted to mate with a corresponding structure of a needle hub, wherein the interlock structure forms an outer thread, which allow the cannula to be threadably inserted into a needle hub.

In another aspect, the products comprises an alternative embodiment of an electroformed needle cannula for an injection device, wherein the cannula comprises:
an electroformed tip having an outer surface, wherein the outer surface defines a plane having a normal vector, and wherein an angle between the cylindrical axis and the normal vector ranges from [0-90] deg or from [30-60] deg.

In another aspect, the products comprises an alternative embodiment of an electroformed needle cannula for an injection device, wherein the cannula comprises:
a first section having a first inner diameter, a second section having a second inner diameter and a transition section, wherein the transition section connects the first and the second section, and wherein the first diameter is different from the second diameter.

In another aspect, the products comprises an alternative embodiment of an electroformed needle cannula for an injection device, wherein the cannula comprises:
a first biocompatible layer of metal or metal alloy, a second layer of metal or metal alloy, and a third biocompatible layer of metal or metal alloy.

In another aspect, the products comprises an alternative embodiment of an electroformed needle cannula for an injection device, wherein the cannula comprising an insertion stop 154, 155 or a pressure fit structure 156, 157, 158.

### Conduction of Experiment:

The experiments were conducted in a galvanic bath 2 as shown on FIG. 1 to FIG. 3. One way of conducting the experiments is described in more details below:

### Preparation

First, the mandrel was cleaned by exposing the mandrel to the following sequence of cleaning agents: DI water, isopropanol, DI water, acetone, DI water, ethanol, DI water. Thereafter the form releasing structure (a POM collar) was attached, and the mandrel was degreased in NaOH, which created a nm thick layer of chrome oxide. The mandrel was submerged into the NaOH and a voltage of 5 V was applied in 2 minutes. The mandrel was rotating.

### Electroforming

The mandrel was immersed into the electrolyte (a sulfamate bath) under rotation, and the current was ramped up from 0 to 41 mA in 45 s. The mandrel was electroplated at a speed of approximately 60 micrometer/hour. The current was turned off, and the mandrel was removed from the bath. Finally, the mandrel was cleaned in DI water, and the cannula was demoulded by pulling in opposite directions of the mandrel and the form releasing structure.

### EXAMPLES

The following cannulas was produced by the above mentioned process

### Example 1

Mandrel: LCP (liquid crystal polymer)/COC (cyclic olefin copolymer)/PPA (polyphthalamide) with Cu deposited by means of CVD (chemical vapour deposition) to obtain conducting properties.

Metal or metal alloy: Ni

The mandrel was electroformed in the dimensions: L (length)=7 mm, WT (wall thickness)˜100 micrometer, IDₚₑ (inner diameter at the patient end)=600 micrometer.

### Example 2

Mandrel: AISI 304 (stainless steel)
Electroforming metal: Ni
Dimensions: L=14 mm, WT˜130 micrometer, IDₚₑ=120 micrometer

### Example 3

Mandrel: AISI 304

Metal or metal alloy: Ni

Dimensions: L=14 mm, WT˜30 micrometer, IDₚₑ=120 micrometer

### Example 4

Mandrel: AISI 304

Metal or metal alloy: SnNi

Dimensions: L=14 mm, WT˜30 micrometer, IDₚₑ=120 micrometer.

### Example 5

Mandrel: AISI 304

Metal or metal alloy: Ni and SnNi

First layer SnNi (1.6 micrometer), second layer Ni (32 micrometer), third layer SnNi (2.2 micrometer). Dimensions: L=14 mm, WT˜35 micrometer, IDₚₑ=100 μm

### List of Embodiments

1. A method of electroforming a needle cannula for an injection device, wherein the electroforming method is performed in an electroforming system comprising a cathode, an anode and an electrolyte with dissolved metal ions, wherein the method comprises:
   providing a permanent mandrel, wherein the mandrel is configured to constitute the cathode, wherein the mandrel is having a forming portion with a forming surface adapted to form the inner surface of the needle cannula, wherein the mandrel comprises a cylindrical axis, a longitudinal extension, a first proximal end and a second distal end,
   electrodepositing a metal or metal alloy on the forming surface of the mandrel, where the electrodeposited metal or metal alloy is corresponding to the metal ions dissolved in the electrolyte, and whereby the electrodeposited metal or metal alloy is forming a needle cannula on the mandrel, and
   separating the mandrel from the formed needle cannula by moving the mandrel and the electroformed needle cannula relative to each other.
2. A method of electroforming a needle cannula according to embodiment 1, wherein the mandrel comprises an aspect ratio above 50, wherein the aspect ratio is defined as the ratio between a longitudinal length of the portion of the mandrel, where a metal or metal alloy is to be deposited, and a largest diameter of said portion of the mandrel.
3. A method of electroforming a needle cannula according to any of embodiments 1-2, wherein the ratio between the surface area of the portion of the mandrel, where a metal or metal alloy is to be deposited, and the volume of said portion is larger than 45×10³ m⁻¹.
4. A method of electroforming a needle cannula according to any of the previous embodiments, wherein the permanent mandrel is intact after separation, and can be used in an electrodeposition process several times.
5. A method of electroforming a needle cannula according to any of the previous embodiments wherein the permanent mandrel comprises stainless steel.
6. A method of electroforming a needle cannula according to any of the previous embodiments wherein the permanent mandrel comprises a thin parting film, wherein the parting film eases the separation process between the mandrel and the electroformed cannula.
7. A method of electroforming a needle cannula according to any of the previous embodiments wherein the permanent mandrel comprises a diameter, wherein the diameter is a constant function or an increasing function of a coordinate defined by the cylindrical axis, and running from the distal end of the mandrel and in the proximal direction, and wherein the diameter function enables the separation of the mandrel and the electroformed needle cannula without entrapping any volume of the mandrel within the formed needle cannula, wherein the method further comprises:
   forming a needle cannula on the mandrel, wherein an inner diameter of the needle cannula is constant function or an increasing function of the coordinate defined by the cylindrical axis.
8. A method of electroforming a needle cannula according to any of the previous embodiments, wherein the mandrel is a tapered cylindrical beam, and whereby the forming surface is conical or frustoconical, wherein the method further comprises:
   forming a needle cannula, wherein an inner surface of the needle cannula has a conical or frustoconical surface.
9. A method of electroforming a needle cannula according to any of embodiments 1-7, wherein the mandrel comprises a first section, a second section, and a transition section connecting the first and the second section,
a. wherein the forming surface comprises a first forming surface corresponding to the first section, a second forming surface corresponding to the second section, and a transition forming surface corresponding to the transition section,
b. wherein the transition forming surface provides a transition surface ensuring a continuous forming surface, when connecting sections with different diameters,
c. wherein the diameter of the mandrel is a constant function or an increasing function of a coordinate defined by the cylindrical axis, and running from the distal end of the mandrel and in the proximal direction,
d. wherein the first spatial derivative of the diameter within the first section is a constant, wherein the first spatial derivate of the diameter within the transition section is a constant, wherein the first spatial derivative of the transition section is larger than the first spatial derivative within the first section, and
e. wherein the method further comprises:
   forming a needle cannula comprising a first section, a second section and a transition section corresponding to the forming surface of the mandrel.
10. A method of electroforming a needle cannula according to embodiment 9, wherein the transition forming surface comprises a normal vector, and an angle between the cylindrical axis and the normal vector, wherein the angle is ranging from [0-90] deg.
11. A method of electroforming a needle cannula according to any of embodiments 9-10, wherein the method further comprises:
   electrodepositing a constant layer on the mandrel, and thereby forming a needle cannula comprising an outer diameter, wherein the outer diameter corresponds to the inner diameter and a constant, and wherein the cannula comprises sections corresponding to the sections of the mandrel.
12. A method of electroforming a needle cannula according to any of embodiments 9-11, wherein a largest diameter of the second section is smaller than a smallest diameter of the first second section, wherein the method further comprises:
   electrodepositing a layer of metal or metal alloy on the mandrel, wherein the cannula comprises sections corresponding to the sections of the mandrel, wherein a largest diameter of the second section is smaller than a smallest diameter of the first section.
13. A method of electroforming a needle cannula according to any of the previous embodiments where the mandrel comprises an electrical non-conducting polymer coated with a thin electrical conducting film.
14. A method of electroforming a needle cannula according to any of the previous embodiments, wherein the mandrel comprises a longitudinal variation of the electrical conductivity, and wherein a deposition rate increases with increased conductivity of the mandrel, and wherein the method further comprises:
   electroforming a needle cannula with a longitudinal variation in the thickness of an electrodeposited layer, where the thickness of the electrodeposited layer corresponds to the electrical conductivity of the mandrel.
15. A method of electroforming a needle cannula according to any of the previous embodiments further comprising:
   depositing a conducting material on the forming surface of the mandrel, and thereby creating an interlock forming structure to form an interlock structure on the needle cannula,
   forming a needle cannula with an interlock structure, and
   separating the needle cannula and the interlock forming structure from the mandrel.
16. A method of electroforming a needle cannula according to embodiment 15 further comprising:
   removing the interlock forming structure from the needle cannula.
17. A method of electroforming a needle cannula according to any of the embodiments 1-15 further comprising:
   depositing a polymer on the forming surface of the mandrel, and thereby creating an interlock forming structure to form an interlock structure on the needle cannula,
   coating the deposited polymer with a conducting film,
   forming a needle cannula with an interlock structure,
   separating the needle cannula and the interlock forming structure from the mandrel, and
   removing the interlock forming structure from the needle cannula.
18. A method of electroforming a needle cannula according to any of the previous embodiments, wherein the anode comprises a metal corresponding to the metal deposited on the mandrel, and thereby the method further comprises
   continuously replenishing the metal used for deposition, by dissolving metal from the anode.
19. A method of electroforming a needle cannula according to any of the previous embodiments, wherein the electroforming system further comprises a form releasing structure, the form releasing structure comprises at least one through hole adapted to receive the mandrel,
   inserting the mandrel into the through hole, whereby the form removing structure is dividing the mandrel into a distal portion and a proximal portion, and wherein the distal portion is to be exposed to the electrolyte, and wherein the proximal portion is to be shielded against the electrolyte,
   immersing the distal portion of the mandrel into the electrolyte,
   separating the mandrel from the formed needle cannula product by moving the mandrel and the form releasing structure in opposite directions.
20. A method of electroforming a needle cannula according to embodiment 19 wherein the form releasing structure comprises a non-conducting hard polymer.
21. A method of electroforming a needle cannula according to any of embodiments 19-20, wherein the form releasing structure comprises a proximal form giving structure having a forming surface for forming a structure at the proximal end of the needle, and whereby the method further comprises:
   electroforming a needle cannula with a structure corresponding to the forming surface of the form giving structure.
22. A method of electroforming a needle cannula according to embodiment 21, wherein the forming surface of the proximal form giving structure is adapted to form an outer surface and/or a proximal tip structure of the electroformed cannula.
23. A method of electroforming a needle cannula according to any of embodiments 21-22, wherein the forming surface of the proximal form giving structure is adapted to form an interlock structure or a thread on an outer surface of the electroformed cannula.
24. A method of electroforming a needle cannula according to any of embodiments 21-23, wherein the forming surface of the proximal form giving structure comprises a conducting film coating, and wherein the forming surface of the proximal form giving structure constitutes a cathode of the electroforming system.
25. A method of electroforming a needle cannula according to embodiment 24, wherein the conducting film coating is adapted to be separated from the forming surface of the proximal form giving structure, and wherein the method further comprises:
   separating the electroformed needle cannula from the forming surface of the proximal form giving structure, by separating the conducting film from the forming surface of the proximal form giving structure.
26. A method of electroforming a needle cannula according to any of embodiments 21-25, whereby the proximal form giving structure comprises a channel to allow a fluid flow and to allow the electrolyte and the dissolved metal ions to replenish the metal deposited at the proximal end of the needle cannula.
27. A method of electroforming a needle cannula according to any of the previous embodiments, wherein the electroforming system further comprises a local form giving structure having a forming surface for forming a structure on the needle cannula, and whereby the method further comprises:
   positioning the local form giving structure at a desired position relative to the mandrel,
   electroforming a needle cannula with a structure corresponding to the forming surface of the local form giving structure.
28. A method of electroforming a needle cannula according to embodiment 27 wherein the local form giving structure comprises a non-conducting hard polymer.
29. A method of electroforming a needle cannula according to any of embodiments 27-28, wherein the forming surface of the local form giving structure is adapted to form an outer surface and/or a distal tip structure of the electroformed cannula.
30. A method of electroforming a needle cannula according to any of embodiments 27-29, wherein the forming surface of the local form giving structure is adapted to form an interlock structure or a thread on an outer surface of the electroformed cannula.
31. A method of electroforming a needle cannula according to any of embodiments 27-30, wherein the forming surface of the local form giving structure comprises a conducting film coating, and wherein the forming surface of the local form giving structure constitutes a cathode of the electroforming system.
32. A method of electroforming a needle cannula according to embodiment 31, wherein the conducting film coating is adapted to be separated from the forming surface of the local form giving structure, and wherein the method further comprises:
   separating the electroformed needle cannula from the forming surface of the local form giving structure, by separating the conducting film from the forming surface of the local form giving structure.
33. A method of electroforming a needle cannula according to any of embodiments 26-32, whereby the local form giving structure comprises a channel to allow a fluid flow and to allow the electrolyte and the dissolved metal ions to replenish the metal deposited at the position of the local form giving structure.
34. A method of electroforming a needle cannula according to any of the previous embodiments, wherein the electroforming system further comprises a holding device comprising a local anode, wherein the local anode is adapted to locally increase the deposition rate, and wherein the method further comprises
   positioning the local anode at a desired position relative to the mandrel,
   electroforming a needle cannula, wherein the electrodeposition rate is increased at an area in the proximity of local anode, and thereby forms an interlock structure on the needle cannula.
35. A method of electroforming a needle cannula according to embodiment 34, wherein the holding device comprises a channel to allow a fluid flow and to allow the electrolyte and the dissolved metal ions to replenish the metal deposited at the position of the local anode.
36. A method of electroforming a needle cannula according to any of embodiments 34-35, wherein the anode comprises a metal corresponding to the metal deposited on the mandrel, and thereby the method further comprises:
   continuously replenishing the metal used for deposition, by dissolving metal from the anode.
37. A method of electroforming a needle cannula according to any of the previous embodiments, wherein the preferred metals to be formed by electrodeposition is Cr, Mn, Tc, Re, Fe, Ru, Os, Co, Rh, Ir, Ni, Pd, Pt, Cu, Ag, Au, Zn, Cd, Hg, In, Tl, Sn, Pb, As, Sb, Bi, Se or Te.
38. A method of electroforming a needle cannula according to any of the previous embodiments, wherein the electrodeposited metal alloy comprises cobalt based alloys, nickel based alloys, iron based alloys, gold based alloys, silver based alloys or platinum based alloys.
39. A method of electroforming a needle cannula according to any of the previous embodiments, wherein the electrodeposited metal alloy comprises CoNi, CoCrMo, CoSn, NiSn, NiW, NiTi, FeNiCr, FeCr, FeNiCrMo or PtIr
40. A method of electroforming a needle cannula according to any of the previous embodiments, wherein the electrodeposited metal alloy comprises one of the elements V, Nb, Mo, W, B, Al, Ge or P.
41. A method of electroforming a needle cannula according to any of the previous embodiments, wherein the system for electroforming comprises a first, and a second electrolyte, wherein the first electrolyte comprises a first solution of metal ions, wherein the second electrolyte comprises a second solution of metal ions, wherein the method further comprising:
   electrodepositing a first metal or metal alloy corresponding to the metal ions of the first solution onto the mandrel,
   electrodepositing a second metal or metal alloy corresponding to the metal ions of the second solution onto the mandrel and/or the first metal or metal alloy.
42. A method of electroforming a needle cannula according to any of the previous embodiments, wherein the system for electroforming comprises a first, and a second electrolyte, wherein the first electrolyte comprises a first solution of metal ions, wherein the second electrolyte comprises a second solution of metal ions, wherein the method further comprises
   electrodepositing a first layer of metal or metal alloy corresponding to the metal ions of the first solution onto the mandrel,
   electrodepositing a second layer of metal or metal alloy corresponding to the metal ions of the second solution onto the first layer of metal or metal alloy, and thereby forming a composite structure.
43. A method of electroforming a needle cannula according to embodiment 42, wherein the method further comprises:
   electrodepositing a third layer of metal or metal alloy onto the second layer of metal or metal alloy, wherein the metal of the third layer corresponds to the metal ions of the first solution or a third electrolyte comprising a third solution of metal ions, and thereby forming a composite structure, where the second layer is substantially covered by the first and the third layer.
44. A method of electroforming a needle cannula according to embodiment 41, wherein the first metal or metal alloy and/the second metal or metal alloy is electrodeposited at the needle cannula tip, and thereby reinforcing the formed tip in order to reduce the tendency of hooking.
45. A method of electroforming a needle cannula according to any of the previous embodiments, wherein the method further comprises:
   using additives, electrochemical feedback or pulse power and thereby reducing stress in the electroformed metal or metal alloy.
46. A method of electroforming a needle cannula according to any of the previous embodiments, wherein the method further comprises:
   optimizing an applied voltage in order to optimize quality and production speed.
47. A method of electroforming a needle cannula according to any of the previous embodiments, wherein the method further comprises:
   performing a finishing process on the electroformed cannula, where the finishing process comprises grinding of the needle tip.
48. A method of electroforming a needle cannula according to any of the previous embodiments further comprising rinsing and degreasing the mandrel before electrodeposition.
49. An electroformed needle cannula for an injection device, wherein the needle cannula is formed by a method according to any of the previous embodiments.
50. An electroformed needle cannula for an injection device according to embodiment 49, wherein the cannula comprises:
   A wall thickness ranging from [20-150] micrometer or more preferably from [30-70] micrometer.
51. An electroformed needle cannula for an injection device according to embodiment 49, wherein the cannula comprises:
   An outer diameter of the distal tip ranging from [120-270] micrometer or more preferably from [170-270] micrometer.
52. An electroformed needle cannula for an injection device according to embodiment 49, wherein the cannula comprises:
   a first section having a first outer diameter, a second section having a second outer diameter and a transition section, wherein the transition section connects the first and the second section, and wherein the first outer diameter is different from the second outer diameter, wherein the transition section is adapted to define an insertion stop, for a cannula inserted into a needle hub.
53. An electroformed needle cannula for an injection device according to embodiment 49, wherein the cannula comprises:
   a first section having a first diameter, a second section having a second diameter and a transition section, wherein the transition section connects the first and the second section, and wherein the first diameter is different from second diameter, wherein the surface of the transition section is tapered and thereby allows the cannula to be pressure fitted, when inserted into a needle hub.
54. An electroformed needle cannula for an injection device according to embodiment 49, wherein the cannula comprises:
   an interlock structure adapted to mate with a corresponding structure of a needle hub, and thereby allowing a cannula to snap fit, when inserted into a needle hub,
   wherein the interlock structure forms a protrusion on the outer surface of the needle cannula, or wherein the interlock structure forms a constriction in the outer surface.
55. An electroformed needle cannula for an injection device according to embodiment 49, wherein the cannula comprises:
   an interlock structure adapted to mate with a corresponding structure of a needle hub, wherein the interlock structure forms an outer thread, which allow the cannula to be threadably inserted into a needle hub.
56. An electroformed needle cannula for an injection device according to embodiment 49, wherein the cannula comprises:
   an electroformed tip having an outer surface, wherein the outer surface defines a plane having a normal vector, and wherein an angle between the cylindrical axis and the normal vector ranges from [0-90] deg or from [30-60] deg.
57. An electroformed needle cannula for an injection device according to embodiment 49, wherein the cannula comprises:
   a first section having a first inner diameter, a second section having a second inner diameter and a transition section, wherein the transition section connects the first and the second section, and wherein the first diameter is different from the second diameter.
58. An electroformed needle cannula for an injection device according to embodiment 49, wherein the cannula comprises:
   a first biocompatible layer of metal or metal alloy, a second layer of metal or metal alloy, and a third biocompatible layer of metal or metal alloy.
59. A needle assembly for an injection device comprising:
   a needle cannula according to any of embodiments 49-58, and
   a needle hub adapted to receive the cannula, and wherein the needle hub is having attachment means for attachment to an injection device.
60. A needle assembly according to embodiment 59, wherein the needle hub further comprises an interlock structure, wherein the needle cannula comprises an interlock structure adapted to mate with the corresponding interlock structure of the hub.
61. A needle assembly according to embodiment 59, wherein the needle hub further comprises a threaded surface, wherein the needle cannula comprises a threaded surface adapted to mate with the corresponding thread of the hub.
62. A needle assembly according to embodiment 59, wherein the needle hub further comprises a surface to form an insertion stop, wherein the needle cannula comprises a transition section having a surface, wherein the surface is forming an insertion stop adapted to mate with the corresponding surface of the hub.
63. A method of forming a plurality of needle cannulas according to embodiment 49-58 comprising:
   forming a plurality of needle cannulas by using the method according to any of embodiments 1-47 to process the mandrels in parallel.
64. A method of electroforming a needle cannula for an injection device, wherein the electroforming method is performed in an electroforming system comprising a cathode, an anode and an electrolyte with dissolved metal ions, wherein the method comprises:
   providing a permanent mandrel, wherein the mandrel is configured to constitute the cathode, wherein the mandrel is having a forming portion with a forming surface adapted to form the inner surface of the needle cannula, wherein the mandrel comprises a cylindrical axis, a longitudinal extension, a first proximal end and a second distal end,
   electrodepositing a metal or metal alloy on the forming surface of the mandrel, where the electrodeposited metal or metal alloy is corresponding to the metal ions dissolved in the electrolyte, and whereby the electrodeposited metal or metal alloy is forming a needle cannula on the mandrel,
   separating the mandrel from the formed needle cannula by moving the mandrel and the electroformed needle cannula relative to each other,
   forming an interlock structure.
65. A method of electroforming a needle cannula according to embodiment 64, wherein the electroforming system further comprises a holding device (70) comprising a local anode (71), wherein the local anode (71) is adapted to locally increase the deposition rate, and wherein the method further comprises:
   positioning the local anode (71) at a desired position relative to the mandrel,
   electroforming a needle cannula, wherein the electrodeposition rate is increased at an area in the proximity of local anode, and thereby forms an interlock structure (105) on the needle cannula.
66. A method of electroforming a needle cannula according to embodiment 64 further comprising:
   depositing a conducting material on the forming surface of the mandrel, and thereby creating an interlock forming structure (80) to form an interlock structure on the needle cannula,
   forming a needle cannula (100) with an interlock structure (107), and
   separating the needle cannula (100) and the interlock forming structure (80) from the mandrel (10).
67. A method of electroforming a needle cannula according to embodiment 66 further comprising:
   removing the interlock forming structure from the needle cannula.
68. A method of electroforming a needle cannula according to embodiment 64 comprising:
   depositing a polymer on the forming surface of the mandrel, and thereby creating an interlock forming structure to form an interlock structure on the needle cannula,
   coating the deposited polymer with a conducting film,
   forming a needle cannula with an interlock structure,
   separating the needle cannula and the interlock forming structure from the mandrel, and
   removing the interlock forming structure from the needle cannula.
69. A method of electroforming a needle cannula according to embodiment 64, wherein the electroforming system further comprises a local form giving structure (40) having a forming surface (42, 43) for forming a structure on the needle cannula, and whereby the method further comprises:
   positioning the local form giving structure (40) at a desired position relative to the mandrel (10),
   electroforming a needle cannula with a structure corresponding to the forming surface (42, 43) of the local form giving structure (40), wherein the forming surface of the local form giving structure is adapted to form an interlock structure or a thread on an outer surface of the electroformed cannula.
70. An electroformed needle cannula for an injection device obtainable by the method according to any of the embodiments 64-69, wherein the cannula comprises:
   an interlock structure adapted to mate with a corresponding structure of a needle hub, and thereby allowing a cannula to snap fit, when inserted into a needle hub.
71. An electroformed needle cannula according to embodiment 70, wherein the interlock structure forms a protrusion on the outer surface of the needle cannula, or wherein the interlock structure forms a constriction in the outer surface.

While certain features of the invention have been illustrated and described herein, many modifications, substitutions, changes, and equivalents will now occur to those of ordinary skill in the art. It is, therefore, to be understood that the appended examples are intended to cover all such modifications and changes as fall within the true spirit of the invention.

1. A method of electroforming a needle cannula for an injection device, wherein the electroforming method is performed in an electroforming system comprising a cathode, an anode and an electrolyte with dissolved metal ions, wherein the method comprises:
providing a permanent mandrel, wherein the mandrel is configured to constitute the cathode, wherein the mandrel is having a forming portion with a forming surface adapted to form the inner surface of the needle cannula, wherein the mandrel comprises a cylindrical axis, a longitudinal extension, a first proximal end and a second distal end,
electrodepositing a metal or metal alloy on the forming surface of the mandrel, where the electrodeposited metal or metal alloy is corresponding to the metal ions dissolved in the electrolyte, and whereby the electrodeposited metal or metal alloy is forming a needle cannula on the mandrel,
separating the mandrel from the formed needle cannula by moving the mandrel and the electroformed needle cannula relative to each other, wherein the system for electroforming comprises a first, and a second electrolyte, wherein the first electrolyte comprises a first solution of metal ions, wherein the second electrolyte comprises a second solution of metal ions, wherein the method further comprises:
electrodepositing a first layer of metal or metal alloy corresponding to the metal ions of the first solution onto the mandrel,
electrodepositing a second layer of metal or metal alloy corresponding to the metal ions of the second solution onto the mandrel and/or the first metal or metal alloy.

2. A method of electroforming a needle cannula according to claim 1, wherein the method further comprises
forming a composite structure, by electrodeposition of the second layer onto the first layer.

3. A method of electroforming a needle cannula according to claim 1, wherein the method further comprises:
electrodepositing a third layer of metal or metal alloy onto the second layer of metal or metal alloy, wherein the metal of the third layer corresponds to the metal ions of the first solution or a third electrolyte comprising a third solution of metal ions, and thereby forming a composite structure, where the second layer is substantially covered by the first and the third layer.

4. A method of electroforming a needle cannula according to claim 1, wherein the first layer of metal or metal alloy and the second layer of metal or metal alloy is electrodeposited at the needle cannula tip, and thereby reinforces the formed tip in order to reduce the tendency of hooking.

5. An electroformed needle cannula for an injection device, obtainable by a method according to claim 1, wherein the cannula comprises:
a first layer of metal or metal alloy, and a second layer of metal or metal alloy, wherein the second layer reinforces the cannula tip.

6. An electroformed needle cannula for an injection device, obtainable by a method according to claim 1, wherein the cannula comprises:
a first layer of metal or metal alloy, and a second layer of metal or metal alloy, wherein the outer surface of the needle cannula is covered by the second layer.

7. An electroformed needle cannula according to claim 6, wherein the second layer is a biocompatible outer layer.

8. An electroformed needle cannula for an injection device, wherein the needle cannula is formed by a method according to claim 1, wherein the cannula comprises:
a first biocompatible layer of metal or metal alloy, a second layer of metal or metal alloy, and a third biocompatible layer of metal or metal alloy.

9. A needle cannula according to claim 8, wherein the second layer is covered by the first and the third layer.

10. A method of electroforming a needle cannula for an injection device, wherein the electroforming method is performed in an electroforming system comprising a cathode, an anode and an electrolyte with dissolved metal ions, wherein the method comprises:
providing a permanent mandrel, wherein the mandrel is configured to constitute the cathode, wherein the mandrel is having a forming portion with a forming surface adapted to form the inner surface of the needle cannula, wherein the mandrel comprises a cylindrical axis, a longitudinal extension, a first proximal end and a second distal end,
electrodepositing a metal or metal alloy on the forming surface of the mandrel, where the electrodeposited metal or metal alloy is corresponding to the metal ions dissolved in the electrolyte, and whereby the electrodeposited metal or metal alloy is forming a needle cannula on the mandrel,
separating the mandrel from the formed needle cannula by moving the mandrel and the electroformed needle cannula relative to each other,
forming an interlock structure.

11. A method of electroforming a needle cannula according to claim 10, wherein the electroforming system further comprises a holding device comprising a local anode, wherein the local anode is adapted to locally increase the deposition rate, and wherein the method further comprises:
positioning the local anode at a desired position relative to the mandrel,
electroforming a needle cannula, wherein the electrodeposition rate is increased at an area in the proximity of local anode, and thereby forms an interlock structure on the needle cannula.

12. A method of electroforming a needle cannula according to claim 10 further comprising:
depositing a conducting material on the forming surface of the mandrel, and thereby creating an interlock forming structure to form an interlock structure on the needle cannula,
forming a needle cannula with an interlock structure, and
separating the needle cannula and the interlock forming structure from the mandrel.

13. A method of electroforming a needle cannula according to claim 12 further comprising:
removing the interlock forming structure from the needle cannula.

14. A method of electroforming a needle cannula according to claim 10 further comprising:
depositing a polymer on the forming surface of the mandrel, and thereby creating an interlock forming structure to form an interlock structure on the needle cannula,
coating the deposited polymer with a conducting film,
forming a needle cannula with an interlock structure,
separating the needle cannula and the interlock forming structure from the mandrel, and
removing the interlock forming structure from the needle cannula.

15. An electroformed needle cannula for an injection device obtainable by the method according to claim 10, wherein the cannula comprises:
an interlock structure adapted to mate with a corresponding structure of a needle hub, and thereby allowing a cannula to snap fit, when inserted into a needle hub,
wherein the interlock structure forms a protrusion on the outer surface of the needle cannula.
